SEQUENCE LISTING (4 sequences)

SEQ ID NO: 1 - DNA, 20 bp, artificial sequence
  primer for PCR
cgtgagggcatcgaggtggc

SEQ ID NO: 2 - DNA, 20 bp, artificial sequence
  primer for PCR
gcgtaggcgtcggtcacaaa

SEQ ID NO: 3 - DNA, 18 bp, artificial sequence
  primer for PCR, biotin-labeled at 5′ end
agatgcaccgtcgaacgg

SEQ ID NO: 4 - DNA, 19 bp, artificial sequence
  primer for PCR, biotin-labeled at 5′ end
gccacgtaggcgaaccctg

(19) United States
(12) Patent Application Publication
(10) Pub. No.: US 2008/0254458 A1
(43) Pub. Date: Oct. 16, 2008

(54) Method of pre-treating in pleural effusion for detection of Mycobacterium tuberculosis

(75) Inventor: George Chin-Sheng Chou, Hsin-Shi (TW)

Correspondence Address: John G. Chupa;Law Offices of John Chupa & Associates, P.C., Suite 50, 28535 Orchard Lake Road, Farmington Hills, MI, 48334, US

(73) Assignee: AsiaGen Corporation

(21) Appl. No.: 11/784,822

(22) Filed: Apr. 10, 2007

(51) Int. Cl. C12Q 1/68; C12Q 1/37

(52) U.S. Cl. 435/6; 435/23

(57) ABSTRACT

The present invention provides a method for preparing nucleic acid from a sample for detecting Mycobacterium comprises: (a) reducing the viscosity and decontaminating the sample by a reagent; (b) lysing the sample by adding an alkali solution; and (c) adjusting pH value by adding a pH-adjusting solution. Said method further comprises a method for concentrating the nucleic acid by using a nucleic acid-purifying column together with/without adding a protease into the treated sample.

The present invention also provides a kit for preparing nucleic acid from a sample for detecting Mycobacterium comprises: (a) a solution of N-Acetyl-L-Cystein/Citrate/Sodium Hydroxide (NALC-Citrate-NaOH); (b) an alkali solution; and (c) a pH-adjusting solution. Said kit further comprises a nucleic acid-purifying column together with/without a protease.

### FIELD OF THE INVENTION

The present invention relates to a method for pre-treating a sample for enhancing the sensitivity of detecting Mycobacterium.

### BACKGROUND OF THE INVENTION

Tuberculosis (TB) remains the number one cause of infectious diseases related deaths worldwide. At the same time an increasing number of atypical presentations of AIDS patients with Mycobacterium tuberculosis (MTB) are also seen, with a vast increase in extra pulmonary diseases (Persing, D. H., J. Clin. Microbiol. 29:1281-1285, 1991). Moreover, of particular importance, is the emergence of MTB multi drug-resistant, which causes over 90% mortality in immunocompromised hosts (Shankar, P., et al. Lancet 335:423, 42.1990).

M. bovis and M. africanum which are closely related organisms to MTB also can infect human beings (Young L S. Clin Infect Dis 1993; 17:8436-8441). In addition, there are still other important human pathogens belonging to the genus Mycobacterium which are related to MTB, such as M. avium and M. leprae.

Tuberculosis primarily affects lungs (Pulmonary tuberculosis), although in one-third of the cases other organs are also affected. It is mainly transmitted by patients with infectious pulmonary tuberculosis in the form of airborne droplet produced during coughing and sneezing.

Tuberculous pleurisy (affecting the membranes around the lungs) leads to an accumulation of fluid in the pleural cavity (the normally very small space between the membranes) and partial collapse of the lung.

The conventional diagnosis of tuberculosis based on the golden standard diagnostic process, culture-grown organisms, is extremely sensitive (10-100 organisms per ml present for growth) but is expensive and time-consuming (2-8 weeks), because of the slow doubling rate of acid-fast bacilli (AFB) (Vaneechoutte M., et al. J. Clin. Microbiol. 31:2061-2065. 1993). Direct staining procedures are rapid but lack sensitivity and specificity since Legionella and Nocardia are stained as well (Rogall, T., et al. Int. J. Syst. Bacteriol. 40:323-330, 1990). A preliminary chest X-ray is also routinely used to diagnose pleural MTB.

By overcoming the inherent limitations of stain and culture techniques, molecular methods have great potential for the rapid diagnosis of Mycobacterium tuberculosis. The polymerase chain reaction (PCR) was reported (Cousins, D. V., et al. J. Clin. Microbiol. 30:255-258, 1992) to be useful in the direct diagnosis of tuberculosis infection directly from a variety of clinical specimens (Bocart, D., et al. Am Rev Respir Dis 145:1142-1148, 1992) with a diversity of genetic elements used as target templates. The rapid detection of MTB by PCR and the high degree of sensitivity in clinical samples afforded by DNA amplification have been described by various investigators (Cousins, D. V., et al. J. Clin. Microbiol. 30:255-258, 1992 Del Portillo, P., et al. J. Clin. Microbiol. 29:2163-2168, 1991; Folgueira, L., et al. J. Clin. Microbiol. 31:1019-1021, 1993). PCR analysis of chromosomal DNA is a simple and rapid method for identifying MTB to the species level and in clinical specimens (Shawar, R. M., et al. J. Clin. Microbiol. 31:61-65, 1993).

Sample preparation is a critical step of detecting MTB using molecular biology method. This invention relates an improved method of preparing pleural effusion sample for a fast and accurate diagnostic of MTB using molecular biology methods.

### SUMMARY OF THE INVENTION

The present invention provides a method for preparing nucleic acid from a sample for detecting Mycobacterium comprises:
(a) reducing the viscosity and decontaminating the sample by a reagent;
(b) lysing the sample by adding an alkali solution; and
(c) adjusting pH value by adding a pH-adjusting solution.
   Said method further comprises a method for concentrating the nucleic acid by using a nucleic acid-purifying column together with/without adding a protease into the treated sample.

The present invention also provides a kit for preparing nucleic acid from a sample for detecting Mycobacterium comprises:
(a) a solution of N-Acetyl-L-Cystein/Citrate/Sodium Hydroxide (NALC-Citrate-NaOH);
(b) an alkali solution; and
(c) a pH-adjusting solution.
   Said kit further comprises a nucleic acid-purifying column together with/without a protease.

### BRIEF DESCRIPTION OF THE DRAWINGS

FIG. 1 shows the PCR result of pleural effusion samples collected from suspicious TB patients. M: Marker; No. 1˜17: clinical samples; No. 18: P-PC, pleural effusion positive control and No. 19: P-NC, pleural effusion negative control.

FIG. 2 shows PCR result of the samples after pH adjusting. M: Marker; PC: pleural effusion positive control; NC: pleural effusion negative control; S-PC: C66/sputum TB positive control and S-NC: C66/sputum negative control. (a) shows the result of clinical samples No. 8 and 16 and (b) shows the result of all clinical samples.

FIG. 3 shows PCR results of samples No. 4, 14, 15 and 16 treated by method (1). The labels 1 ul, 3 ul and 5 ul are the sample volume used as PCR template. M: Marker.

FIG. 4 shows PCR results of samples No. 4, 14, 15 and 16. The labels 1 ul, 3 ul and 5 ul are the sample volume used as PCR template. M: marker, lane 1-4: the samples are treated by method (1) and lane 5-6: the samples are treated by method (2).

FIG. 5 shows PCR results of samples No. 4, 14, 15 and 16 concentrated by method (1). Samples No. 15 and 16 were processed through PCR, concentrated by method (2). P-PC was the pleural effusion positive control. PE+PK represents pleural effusion sample treated with proteinase K. M: marker.

FIG. 6 shows PCR results of the samples, which were treated with protease K, passed through column and washed with washing buffer before running on a gel. (a) Samples No. 1, 8, 13 and 17. Each sample was triplicated with starting template volume 1 μl, 3 μl and 5 μl. (b) Samples No. 2, 3, 5, 6, 7, 9, 10, 11 and 12. Each sample had a starting template volume of 5 μl.

### DETAILED DESCRIPTION OF THE INVENTION

The present invention provides a method for preparing nucleic acid from a sample for detecting Mycobacterium comprises:
(a) reducing the viscosity and decontaminating the sample by a reagent;
(b) lysing the sample by adding an alkali solution; and
(c) adjusting pH value by adding a pH-adjusting solution.

The Mycobacterium comprises M. tuberculosis, M. africanum or M. bovis. In a preferred embodiment, the Mycobacterium is M. tuberculosis.

The method of this invention is a pre-treatment method and can remove the mucin and wax layer of Mycobacterium. The sample collected from patient comprises the secretion sample, excrement sample, blood sample, tissue sample, or tissue extract. The secretion sample includes sputum, saliva or nasal mucus. The tissue extract is extracted from tissue such as pleural effusion, gastric juices or interstitial fluid. In the preferred embodiment, the sample is pleural effusion.

In the step (a), the reagent is Hydroxide/Citrate/N-Acetyl-L-Cystein (NaOH-Citrate-NALC).

In the step (b), the alkali solution is applied to lysing the Mycobacterium without damaging the nucleic acid. In the embodiment, the alkali solution not limited but selected from hydroxide solution such as sodium hydroxide or potassium hydroxide solution.

In the step (c), the pH value is adjusted to about neutral pH without damaging the nucleic acid. In the embodiment, the pH value is adjusted to pH 7-8. The pH-adjusting solution is not limited but selected from acid solution, such as hydrochloric acid or acetic acid, or buffer, such as phosphate buffered saline (PBS).

The method of this invention further comprises concentrating the nucleic acid after step (c), which is carried out by using a nucleic acid-purifying column. Further, this step comprises using proteinase. In the preferred embodiment, the proteinase is proteinase K.

The methods of further concentrating the sample in the invention are described below.

### Method (1):

(a) passing the pre-treated sample through a column; and
(b) re-dissolving nucleic acid in deionized water.

### Method (2):

(a) treating the sample from residue fraction of method (1) with proteinase K;
(b) passing samples through a column; and
(c) re-dissolving nucleic acid in deionized water.

### Method (3):

(a) treating the pre-treated sample with proteinase K;
(b) passing samples through a column;
(c) washing samples with washing buffer; and
(d) re-dissolving nucleic acid in deionized water.

In the preferred embodiment, the concentrating method is method (3).

This invention also provides a kit for preparing nucleic acid from a sample for detecting Mycobacterium comprising:
(a) a solution of Sodium Hydroxide/Citrate/N-Acetyl-L-Cystein (NaOH-Citrate-NALC);
(b) an alkali solution; and
(c) a pH-adjusting solution.

The Mycobacterium comprises M. tuberculosis, M. africanum or M. bovis.

In the preferred embodiment, the Mycobacterium is M. tuberculosis.

The sample collected from the patient comprises the secretion sample, excrement sample, blood sample, tissue sample, or tissue extract sample. The secretion sample includes sputum, saliva or nasal mucus. The tissue extract is extracted from tissue such as pleural effusion, gastric juices or interstitial fluid. In the preferred embodiment, the sample is pleural effusion.

The alkali solution of the kit is applied to lysing the Mycobacterium without damaging the nucleic acid. In the embodiment, the alkali solution not limited but selected from hydroxide solution such as sodium hydroxide or potassium hydroxide solution.

The pH value is adjusted to about neutral pH without damaging the nucleic acid. In the embodiment, the pH value is adjusted to pH 7-8. The pH-adjusting solution is not limited but selected from acid solution, such as hydrochloric acid or acetic acid, or buffer, such as phosphate buffered saline.

In this invention, the kit further comprises a column to concentrate the nucleic acid. The kit further comprises a proteinase. In the preferred embodiment, the proteinase is proteinase K.

The invention is illustrated by the following non-limiting examples.

### EXAMPLES

### Example 1

### Preparation of Pleural Effusion Sample

50 ml pleural effusion sample was collected from suspected patient and stored in −20° C. (or −80° C.) until use. The sample was defrosted and centrifuged at 3800 rpm for 10 minutes. The supernatant was removed. 15 ml NaOH-citrate-NALC was added and vortex at room temperature for 15 minutes. PBS was added to total volume 40 ml for neutralizing the sample. The sample was centrifuged at 3800 rpm for 15 minutes and the supernatant was removed. The debris was transferred to a new tube.

Further the sample was centrifuged at 13000 rpm for 2 minutes. 150 μl 0.2 N NaOH was added and vortexed for 1 minute. The sample was placed at room temperature for 10 minutes and then at 100° C. for 20 minutes.

The sample was cooling to room temperature. 125 μl 0.3 M acetic acid solution was added and vortexed for 1 minute. The sample was then centrifuged at 13000 rpm for 2 minutes. The supernatant was collected and retained for further analysis.

### Example 2

### Polymerase Chain Reaction

PCR was performed as well-known in the art. A two-step PCR was performed to detect the Mycobacterium tuberculosis in 18 samples from suspected patients. The procedure was as follows:

### 1. First Step PCR

The following materials were added into a PCR tube:

| | | |
|---|---|---|
| Sample DNA | 1 | ul |
| 10X extension buffer | 5 | ul |
| dNTP | 1 | ul |
| Taq DNA polymerase (5 U/ul) | 0.4 | ul |
| ddH2O | 32.6 | ul |
| Primer A1: | 5 | ul |
| 5′-CGTGAGGGCATCGAGGTGGC-3′ | | |
| Primer A2: | 5 | ul |
| 5′-GCGTAGGCGTCGGTCACAAA-3′ | | |

The amplification cycle was as follows:

| Temperature | | |
|---|---|---|
| (° C.) | Time | Number of cycles |
| 95 | 5 min | 1 cycle |
| 95 | 30 sec | 27 cycles |
| 63.7 | 15 sec | |
| 72 | 15 sec | |
| 72 | 5 min | 1 cycle |
| 4 | hold | — |

### 2. Second Step PCR

The following materials were added into a new PCR tube:

| | | |
|---|---|---|
| PCR product from step 1 | 3 | ul |
| 10X extension buffer | 5 | ul |
| dNTP | 1 | ul |
| Taq DNA polymerase (5 U/ul) | 0.4 | ul |
| ddH2O | 32.6 | ul |
| Primer B1: | 5 | ul |
| 5′-Biotin-AGATGCACCGTCGAACGG-3′ | | |
| Primer B2: | 5 | ul |
| 5′-Biotin-GCCACGTAGGCGAACCCTG-3′ | | |

The amplification cycle was as step 1 PCR.

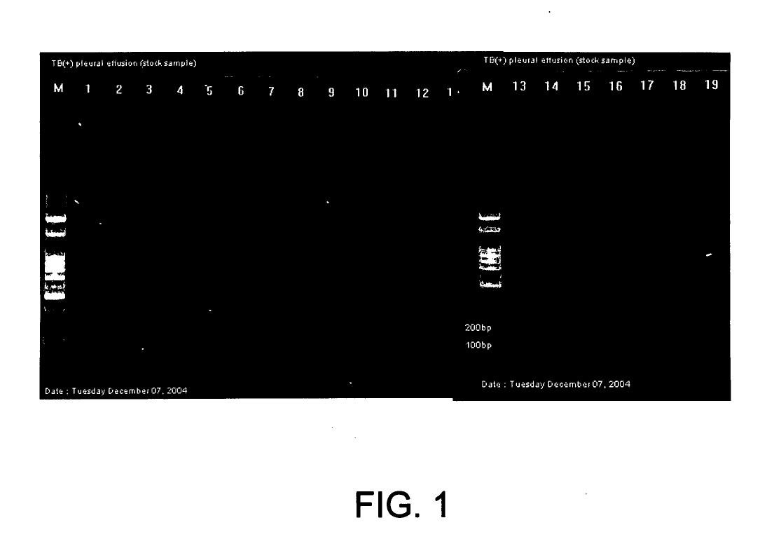

The PCR product was run on a gel as shown in FIG. 1. M: Marker; No. 1˜No. 17: clinical sample/pleural effusion TB(+); No. 18: P-PC (pleural effusion positive control); and No. 19: P-NC (pleural effusion negative control).

The FIG. 1 showed that there was no positive signal of the PCR result. The result might be caused of the extremely basic pH value of the samples. The pH value was determined as pH 14.

### Example 3

### Adjustment of Ph Value of the Sample

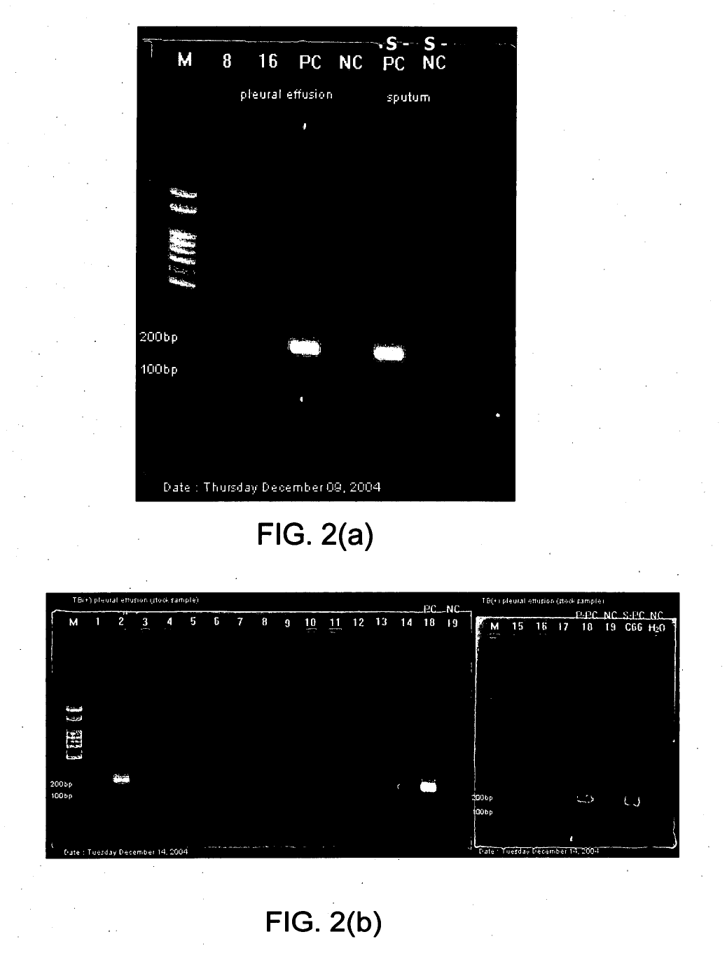

The pH value of the samples No. 1-17 and P-PC and P-NC were adjusted to pH 7-8. The adjusted samples were run on a gel as shown in FIG. 2. The sputum samples from TB patients and normal people were used as the second set of controls. M: Marker; PC: pleural effusion positive control; NC: pleural effusion negative control; S-PC: C66/sputum as positive control and S-NC: C66/sputum as negative control.

The samples No. 2 and No. 14 presented positive signals. The results of sample No. 8 in FIG. 2 (a) (positive) and (b) (negative) did not agree with each other.

### Example 4

### Concentration of DNA by Method (1)

The former negative or discordant results might be caused of the low concentration of the nucleic acid in the sample. Therefore, the nucleic acid of the sample was concentrated before running the PCR product on gel.

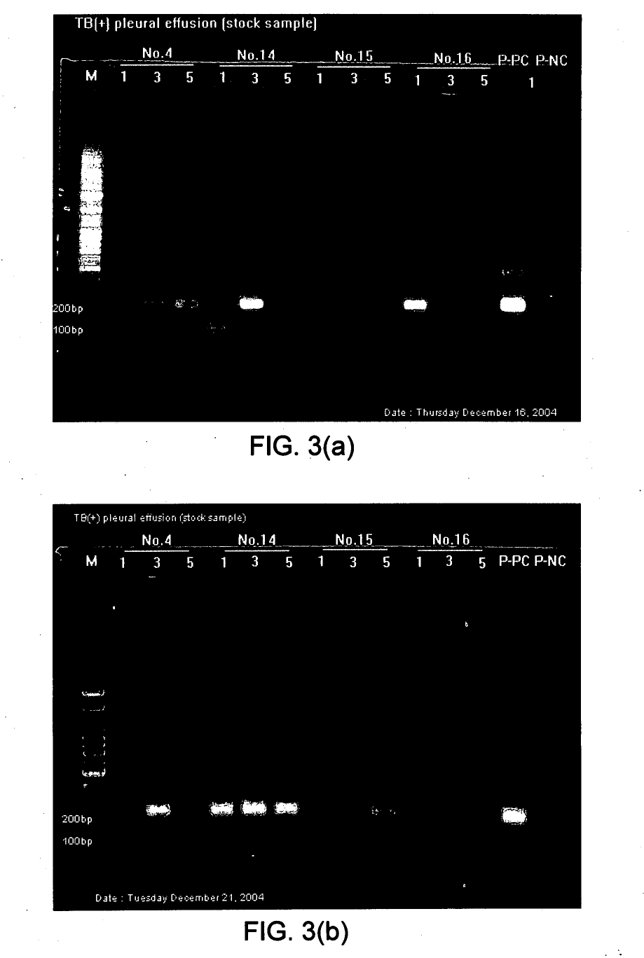

The adjusted samples Nos. 4, 14, 15 and 16 were passed through GeneSpin™ Miniprep column and re-dissolved DNA in 30 μl deionized water. Each sample was used for PCR template and the template volume was 1 ul, 3 ul and 5 ul, respectively. The PCR results were shown in FIG. 3.

The results of FIG. 3 (a) No. 15(-) and FIG. 3 (b) No. 16(-) were discordant. The discordant results might be caused of the low concentrations of M. tuberculosis nucleic acids in the samples. The samples were further concentrated to increase the concentration of DNA.

### Example 5

### Concentration of DNA by Method (2)

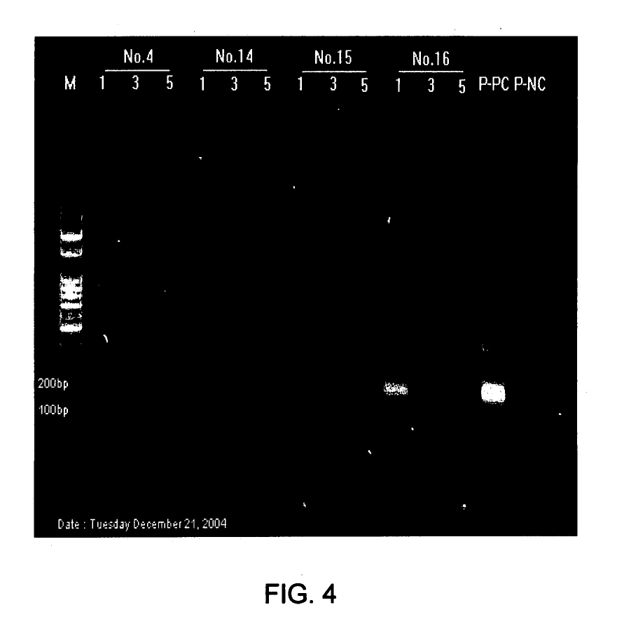

20 μl proteinase K (10 mg/ml) was added to the elution which was collected from example 4. The sample was placed at 55° C. for 45 minutes. The sample was passed through the column and the DNA was re-dissolved in 30 μl deionized water. The result was shown in FIG. 4.

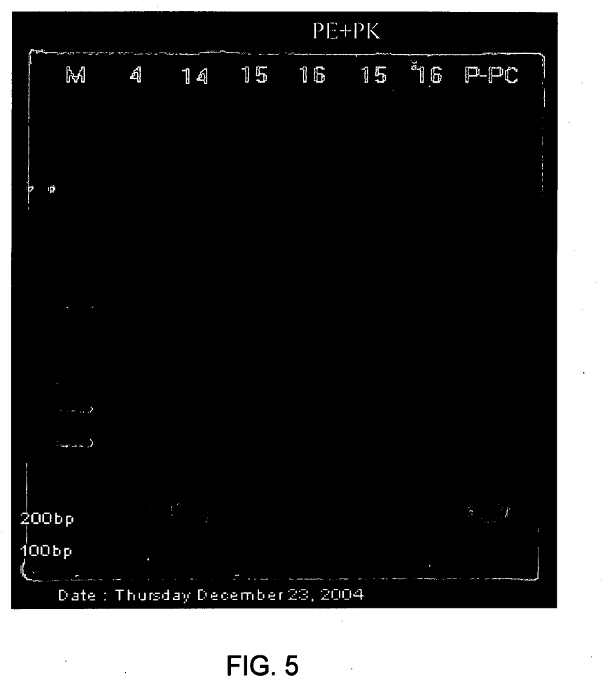

The positive signals of samples No. 4 and No. 16 were observed. The method (1) and method (2) were repeated to compare the efficiency of DNA concentration. Samples Nos. 4, 14, 15 and 16 were processed through PCR and concentrated by using the method (1). Samples No. 15 and 16 were processed through PCR and concentrated by using the method (2). The result was shown in FIG. 5. P-PC was the pleural effusion positive control. PE+PK represented pleural effusion sample treated with proteinase K.

There were positive signals of all samples.

### Example 6

### Concentration of DNA by Method (3)

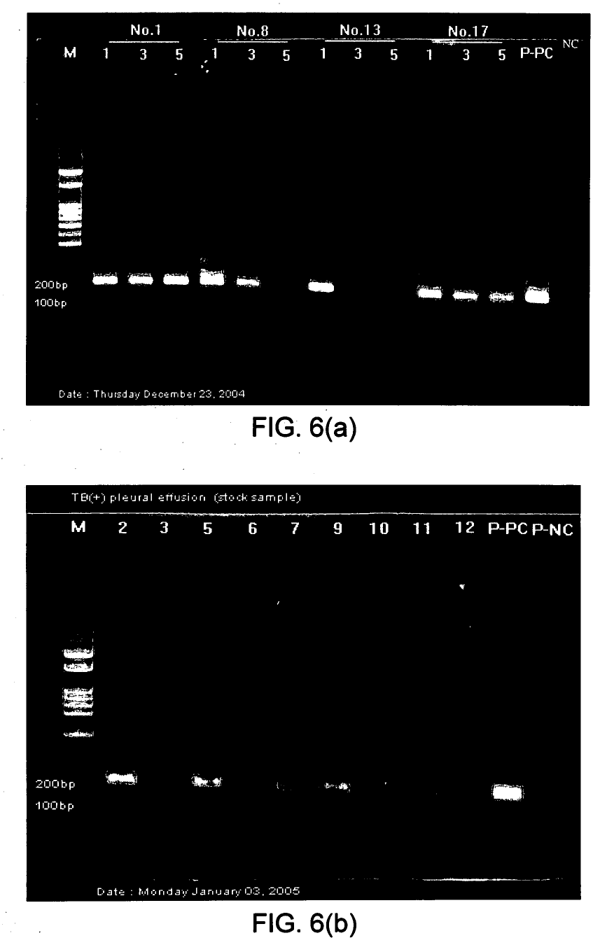

The third method was tried to concentrate the DNA in the sample. 5 μl proteinase K (20 mg/ml) was added to the adjusted sample No. 1, 8, 13 and 17 (pH 7˜8) and placed at 56° C. for 45 minutes. The samples were passed through a GeneSpin™ Miniprep column and washed with the washing buffer included in the GeneSpin™ kit, and re-dissolved in 40 μl deionized water. Each sample was used for PCR template, the template volume was 1 μl, 3 μl and 5 μl, respectively. The results were shown in FIG. 6 (a).

Samples No. 2, 3, 5, 6, 7, 9, 10, 11 and 12 were also concentrated with the same method. The adjusted samples (pH7-8) were treated with 5 μl proteinase K (20 mg/ml) at 56° C. for 45 minutes, passed through columns, washed with washing buffer and re-dissolved in 40 μl deionized water. Each sample was used for PCR template, the template volume was 5 μl. The result was shown in FIG. 6 (b). The positive signals were represented in samples Nos. 2, 3, 5, 7, 9, 11 and 12.

The improved method of pre-treating pleural effusion samples for the detection of Mycobacterium was disclosed. The method was fast, efficient and suitable for large scale application.

### Here is the Sequence List:

Primer A1:
5′-CGTGAGGGCATCGAGGTGGC-3′
Primer A2:
5′-GCGTAGGCGTCGGTCACAAA-3′
Primer B1:
5′-Biotin-AGATGCACCGTCGAACGG-3′
Primer B2:
5′-Biotin-GCCACGTAGGCGAACCCTG-3′

It is to be understood that the present invention is not limited to the exact construction or methodology which has been previously delineated, but that various changes and modifications may be made without departing from the spirit and the scope of the invention as delineated in the following claims.

What is claimed is:

1. A method for preparing nucleic acid from a sample for detecting Mycobacterium comprising:
(a) reducing the viscosity and decontaminating the sample by a reagent;
(b) lysing the sample by adding an alkali solution; and
(c) adjusting pH value by adding a pH-adjusting solution.

2. The method of claim 1, wherein the sample collected from patient comprises the secretion sample, experiment sample, blood sample, tissue sample or tissue extract sample.

3. The method of claim 2, wherein the sample is sputum, saliva, nasal mucus, pleural effusion, gastric juices or interstitial fluid.

4. The method of claim 1, wherein the Mycobacterium comprises M. tuberculosis, M. africanum or M. bovis.

5. The method of claim 1, wherein the reagent is a solution of Sodium Hydroxide/Citrate/N-Acetyl-L-Cystein (NaOH-Citrate-NALC).

6. The method of claim 1, wherein the alkali solution is applied to lysing the Mycobacterium without damaging the nucleic acid.

7. The method of claim 1, wherein the alkali solution is a solution of sodium hydroxide or potassium hydroxide.

8. The method of claim 1, wherein the pH-adjusting solution is an acid solution or a buffer adjusting the pH value to about neutral without damaging the nucleic acid.

9. The method of claim 1, wherein the pH-adjusting solution is hydrochloric acid, acetic acid or phosphate buffer saline.

10. The method of claim 1, which further comprises a method for concentrating the nucleic acid after step (c).

11. The method of claim 10, which is carried out by passing through a nucleic acid-purifying column.

12. The method of claim 10, which further comprises adding proteinase into the nucleic acid sample.

13. A kit for preparing nucleic acid from a sample for detecting Mycobacterium comprising:
(a) a solution of Sodium Hydroxide/Citrate/N-Acetyl-L-Cystein (NaOH-Citrate-NALC);
(b) an alkali solution; and
(c) a pH-adjusting solution.

14. The kit of claim 13, wherein the sample is sputum, saliva, nasal mucus, pleural effusion, gastric juices or interstitial fluid.

15. The kit of claim 13, wherein the Mycobacterium comprises M. tuberculosis, M. africanum or M. bovis.

16. The kit of claim 13, wherein the alkali solution is applied to lysing the Mycobacterium without damaging the nucleic acid.

17. The kit of claim 13, wherein the alkali solution is a solution of sodium hydroxide or potassium hydroxide.

18. The kit of claim 13, wherein the pH-adjusting solution is an acid solution or a buffer adjusting the pH value to about neutral without damaging the nucleic acid.

19. The kit of claim 13, wherein the pH-adjusting solution is hydrochloric acid, acetic acid or phosphate buffer saline.

20. The kit of claim 13, which further comprises a nucleic acid-purifying column.

21. The kit of claim 13, which further comprises a proteinase.
